(19) United States
(12) Patent Application Publication
(10) Pub. No.: US 2020/0027212 A1
(43) Pub. Date: Jan. 23, 2020

(54) METHOD AND DEVICE FOR PRODUCING A PILL BOX

(71) Applicant: DISTRAIMED, Montastruc la Conseillère (FR)

(72) Inventors: Michel FOUBET, Saint-Jean (FR); Olivier FOUBET, Bressols (FR); Grégory FOUBET, Toulouse (FR)

(21) Appl. No.: 16/491,402

(22) PCT Filed: Feb. 8, 2018
(86) PCT No.: PCT/EP2018/053200

§ 371 Date: Sep. 5, 2019

(30) Foreign Application Priority Data
Mar. 10, 2017 (FR) 1751996

(51) Int. Cl. G06T 7/00; G05B 15/02; G16H 20/10; G06T 7/70

(52) U.S. Cl. CPC G06T 7/0014; G05B 15/02; G06T 2207/30242; G06T 7/70; G06T 2207/30004; G16H 20/10

(57) ABSTRACT

The invention relates to a method for preparing a pillbox (23) comprising a plurality of cavities for receiving at least one pill,
in which the following steps are performed:
   at least two filling steps in which, in each filling step, a group of at least one pill is placed in at least one cavity of the pillbox (23) according to data, named filling data,
   a step of acquiring an image of the pillbox (23) by an image acquisition device (24) after at least two filling steps,
characterised in that, after at least one acquisition step, named current acquisition step, a step of checking the filling of the pillbox (23) is performed in which:
   an image acquired during said current acquisition step is compared with an image acquired during an acquisition step preceding said current acquisition step,
   then conformity of the filling is evaluated.

The invention relates to a method for preparing a pillbox comprising a plurality of cavities for receiving at least one medicine, named pill, in divided solid galenic form (i.e., in the solid and divided state e.g., chosen from the group formed by tablets, cachets, gelatine capsules, capsules, pills, pastilles, sugar-coated tablets; or, in contrast, in a solid mass or compact state such as pastes, ointments . . . )—at least not entirely powdery, or even non-powdery—in which at least one pill is placed in at least one cavity of the pillbox according to filling data of a medical prescription for a therapeutic treatment of a patient. The invention also relates to a device for preparing a pillbox.

Within the scope of a therapeutic treatment of a patient, medicines, named pills, in divided solid galenic form can be distributed by a caregiver to the patient according to a medical prescription. This medical prescription indicates in particular the dosage, the frequency at which medicines are to be taken and the duration of the treatment.

In order to facilitate the distribution of the medicines to the patient, the pills to be distributed are generally prepared upstream of the distribution, being gathered together in a package, named pillbox, which may comprise a plurality of cavities for receiving pills (e.g., as described in FR 2 913 333). This preparation step is generally performed manually by a caregiver.

During this preparation step, at least one pill is placed in at least one cavity of the pillbox according to a medical prescription of a single patient. More particularly, the pillbox can be e.g., a daily pillbox or a weekly pillbox.

More particularly, in certain pillboxes, all the pills placed in the same cavity are identical, i.e., they have the same galenic form, the same dosage, the same active substance, the same size . . . .

The preparation step is carried out meticulously and requires concentration and dexterity by the caregiver during manual placement of the pills in the cavities of the pillbox.

In particular, the preparation step must be carried out all the more meticulously when the cavities of the pillbox are small and close together.

When the caregiver does not have both concentration and dexterity, it is possible for pills not to be placed in the right cavities. In fact, it is possible that, if the caregiver is tired and/or inattentive, this person may inadvertently place a pill in the wrong cavity or pick the wrong pill to place. Furthermore, it is possible, during the placement, for a pill to bounce on a wall of a cavity or on another pill and into the wrong cavity.

Now, incorrect placement of the pills in a pillbox can cause errors in administration and thus lead to harmful consequences for the patient after distribution. For example, the treatment can lose its therapeutic efficacy or even lead to the development of side effects prejudicial to the patient.

DE 102011086575 describes a filling method in which a pillbox having a plurality of cavities for receiving pills is placed on a screen. A medicine is then placed in a cavity of a pillbox to be prepared from filling data associated with a patient, then an image of the pillbox is acquired. This image is then displayed on a zone of the screen on which the pillbox is not placed and is then stored in a database. These steps are repeated until all of the cavities of the pillbox are filled.

The method described in DE 102011086575 makes it possible to track the filling of the pillbox by archiving the images in a database. Nevertheless it does not permit the above-mentioned disadvantages to be avoided.

The invention aims to overcome these disadvantages.

It aims in particular to propose a reliable method for preparing a pillbox.

The invention also aims to propose such a preparation method which is simple to implement.

The invention also aims to propose a device for preparing a pillbox adapted to implement such a method.

The invention likewise aims to propose such a device which is inexpensive.

In order to do this, the invention relates to a method for preparing a pillbox comprising a plurality of cavities for receiving at least one medicine, named pill, in divided solid galenic form—at least not entirely powdery, or even non-powdery—

a method in which the following steps are performed:
a step, named initial step, in which the cavities of the pillbox are placed facing an image acquisition device,
at least two filling steps in which, in each filling step, a group of at least one pill is placed in at least one cavity of the pillbox according to data, named filling data, of a medical prescription for a therapeutic treatment of a patient,
at least two steps of image acquisition of the pillbox by the image acquisition device, including:
   a step of acquiring an image of the pillbox after a filling step,
   another step of acquiring an image of the pillbox after another filling step, characterised in that, after at least one—in particular each—acquisition step, named current acquisition step, a computer-implemented step of checking the filling of the pillbox is performed in which:
   an image acquired during said current acquisition step is compared with an image acquired during an acquisition step preceding said current acquisition step so as to identify the pills placed between this acquisition step preceding said current acquisition step and the current acquisition step,
   then conformity of the filling is evaluated from a comparison between the identified pills and the filling data.

The invention relates to a device for preparing a pillbox comprising a plurality of cavities for receiving at least one medicine, named pill, in divided solid galenic form—at least not entirely powdery, or even non-powdery—a preparation device comprising an image acquisition device adapted to:
be placed facing the cavities of the pillbox during at least two filling steps in which, in each filling step, a group of at least one pill is placed in at least one cavity of the pillbox according to data, named filling data, of a medical prescription for a therapeutic treatment of a patient,
perform at least two image acquisitions of the pillbox by the image acquisition device, including:
   acquisition of an image of the pillbox after a filling step,
   another acquisition of an image of the pillbox after another filling step, characterised in that it comprises a data processing device comprising means for implementing, after at least one—in particular each—acquisition step, named current acquisition step, a step of checking the filling of the pillbox in which:
   an image acquired during said current acquisition step is compared with an image acquired during an acquisition step preceding said current acquisition step so as to identify the pills placed between this acquisition step preceding said current acquisition step and the current acquisition step,
   then conformity of the filling is evaluated from a comparison between the identified pills and the filling data.

The invention also relates to a computer program comprising program code instructions for performing the steps of a method in accordance with the invention when said program is run on a computer.

In particular, the computer program comprises program code instructions which, when the program is run by a computer, cause said computer to implement a step of checking filling of a pillbox comprising a plurality of cavities for receiving at least one medicine, named pill, in divided solid galenic form, in which:
an image comprising said pillbox comprising a group, named first group, of at least one pill placed in at least one cavity of the pillbox is compared with an image comprising said pillbox comprising said first group of at least one pill and a group, named second group, of at least one pill placed in at least one cavity of the pillbox so as to identify each pill of said second group,
then conformity of the filling is evaluated from a comparison between the identified pills and data, named filling data.

The invention also relates to a computer-readable recording medium comprising program code instructions for performing the steps of a method in accordance with the invention when they are run on a computer.

In particular, the computer-readable recording medium comprises program code instructions which, when they are run by a computer, cause said computer to implement a step of checking filling of a pillbox comprising a plurality of cavities for receiving at least one medicine, named pill, in divided solid galenic form, in which:
an image comprising said pillbox comprising a group, named first group, of at least one pill placed in at least one cavity of the pillbox is compared with an image comprising said pillbox comprising said first group of at least one pill and a group, named second group, of at least one pill placed in at least one cavity of the pillbox so as to identify each pill of said second group,
then conformity of the filling is evaluated from a comparison between the pills identified and data, named filling data.

More particularly, the pillbox to be filled can be e.g., a single-use pillbox as described in FR 2 913 333. In particular, the pillbox can be used e.g., for a daily or weekly medical prescription.

The medicines to be placed during preparation of the pillbox can be chosen from the group formed in particular by tablets, cachets, gelatine capsules, capsules, pills, pastilles and sugar-coated tablets.

Moreover, the data processing device is preferably a computer, i.e., a computing system, comprising at least one processor able to perform at least the checking step of a method in accordance with the invention. The data processing device could also be a microprocessor, a server . . . .

In particular, the data processing device comprises input/output ports. Furthermore, the data processing device also comprises a memory adapted to store digital data.

The image acquisition device can be a photographic device, a camera, a scanner, in particular adapted to be used as a peripheral of said data processing device and connected to said data processing device.

As it is used in the present text, the expression “computer-readable recording medium” can refer to any device which can contain, store, communicate, propagate or transport a program for use thereof by, or in connection with, a computing system, a terminal, an apparatus or a device for running program code instructions. Such a computer-readable recording medium can be—by way of non-limiting example—a terminal, a device, an apparatus, a system or a magnetic, optical, electromagnetic, infrared, semi-conductor or electronic propagation medium. Certain specific, non-exhaustive examples of a computer-readable medium can be the following: a computer terminal, an electrical connection having one or a plurality of conductors, a mass memory (hard disk, USB stick . . . ), a floppy disk, a random access memory (RAM), a read-only memory (ROM), an erasable programmable read-only memory (EPROM or flash memory), an optical fibre, an optically read memory (a read-only or rewritable compact disk).

In certain advantageous embodiments and in accordance with the invention, during said step of checking the filling of the pillbox:
the image acquired during said current acquisition step is compared with an image acquired during the acquisition step which chronologically precedes said current acquisition step so as to identify the pills placed between this acquisition step which chronologically precedes said current acquisition step and the current acquisition step,
then conformity of the filling is evaluated from a comparison between the identified pills and the filling data.

An acquisition step is preferably performed after each filling step.

Furthermore, a step of checking the filling of the pillbox is performed after each acquisition step.

In such embodiments, replacement of the wrongly placed pills, identified following a nonconformity evaluation of the filling, is simplified.

Nevertheless, in other embodiments it is possible to perform the image acquisition steps only after a certain number of filling steps between each of these image acquisition steps. A step of checking the filling is then performed after each image acquisition step by comparing the image acquired during this image acquisition step with that acquired during the preceding acquisition step.

In certain other embodiments, a step of acquiring an image is performed after each filling step and a step of checking the filling is performed only after a certain number of image acquisition steps by comparing the image, named last image, acquired during one of these image acquisition steps with that acquired during one of these acquisition steps and which precedes the step of acquiring said last image, a plurality of acquisition steps having been able to take place between these two acquisition steps.

In certain other embodiments, the image acquisition steps are performed only after a certain number of filling steps between each of these image acquisition steps and a step of checking the filling is performed only after a certain number of image acquisition steps by comparing the image, named last image, acquired during one of these image acquisition steps with that acquired during one of these acquisition steps and which precedes the step of acquiring said last image, a plurality of acquisition steps having been able to take place between these two acquisition steps.

In certain advantageous embodiments and in accordance with the invention, a plurality of pills are placed in at least one cavity of the pillbox, all the pills placed in the same cavity being identical.

Thus, at the end of a preparation method in accordance with the invention, the group of pills in each cavity of the pillbox comprising a plurality of pills comprises only identical pills (same galenic form, same dosage, same active substance, same size . . . ).

The pillbox preferably comprises a plurality of portions each comprising at least one cavity—in particular a plurality of cavities—each portion being intended to receive a unitary dose of pills, i.e., a group of pills intended to be administered substantially in a single dose to a patient at a given time, said group of pills being in conformity with the dosage of a medical prescription intended for the treatment of at least one pathology affecting this patient. The different cavities of each portion are separated from each other so as to isolate the different medicines of the same unitary dose of pills from each other (as described in FR2913333).

Said portions are preferably detachable portions connected to each other by divisible connections so that a portion can be detached for each unitary dose of pills.

For each filling step, the filling data comprise indications as to the type of pill to be placed (in particular the name of the medicine, the dosage and even the colour and size of the pill), the number of pills to be placed in the pillbox and the cavities (position, number) in which they are to be placed. These filling data are pre-recorded in a memory of said data processing device and associated with an identifier of a patient.

In particular, during the initial step, the identifier of the patient for whom the pillbox is to be prepared is indicated—in particular by a caregiver—to said data processing device so that said data processing device can retrieve from the memory the filling data to be used in the method of preparing the pillbox.

More particularly, advantageously and in accordance with the invention, in each filling step an indication, named filling indication, relating to the filling data for this filling step, is displayed on a screen, named filling assistance screen. Such a filling assistance screen is connected to said data processing device.

Furthermore, in certain advantageous embodiments and in accordance with the invention, the filling assistance screen is disposed facing the cavities of the pillbox and opposite the image acquisition device with respect to the pillbox.

More particularly, the filling assistance screen is placed below the cavities, i.e., opposite apertures providing access to the cavities of the pillbox.

Such a filling assistance screen is thus adapted to indicate the cavities in which at least one pill is to be placed by illuminating, according to a predetermined colour code, regions of the screen which are placed below these cavities. Furthermore, the number of pills to be placed in each of these cavities can also be indicated on these regions of the screen.

Such a filling assistance screen makes it possible to facilitate the filling of the pillbox which can be performed in particular by a caregiver.

In particular, in certain advantageous embodiments and in accordance with the invention, in at least one filling step, a plurality of pills are placed in at least one cavity of the pillbox, the pills of said plurality of pills placed in the pillbox during the same filling step being identical.

Thus, only identical pills are handled—in particular by a caregiver—during each filling step. This makes it possible not only to reduce the risk of error in placing pills but also to reduce the preparation time for the pillbox.

Moreover, the pillbox is preferably fixed to a fixing support arranged so that the cavities of the pillbox face the image acquisition device.

The fixing of the pillbox makes it possible to keep the pillbox in the same position with respect to the image acquisition device which is itself fixed with respect to the fixing support. The checking step is thus facilitated.

Moreover, in certain advantageous embodiments and in accordance with the invention, the initial step comprises a step of acquiring an image, named initial image, of the pillbox by the image acquisition device after having placed the pillbox facing the image acquisition device.

During this initial step, the pillbox is preferably empty of medicine.

Nevertheless, in other embodiments, in particular when the same type of pillbox is used for a plurality of pillbox preparation methods, the initial image of the pillbox is recorded in a memory after a first acquisition of this initial image so as to be able to be re-used for all the methods of preparing a pillbox and to avoid the acquisition of a new initial image for each of these methods.

The image acquired during the first filling step following the initial step is compared with the initial image during the checking step succeeding this first filling step.

In certain embodiments, when a filling step is terminated, it is indicated to the data processing device by an input peripheral connected to said data processing device that the filling step has been terminated in order to be able to pass to the acquisition step then to the checking step.

Alternatively, a scale can be placed below the pillbox so as to detect the end of the filling of a filling step according to the weight of the pillbox and a weight of the medicines which is indicated in the filling data so as to pass automatically to the acquisition step and then to the checking step.

During each comparison step, in particular digital data, especially colorimetric data, representative of said image acquired during said current acquisition step are compared with digital data, especially colorimetric data, representative of said image acquired during an acquisition step preceding said current acquisition step. This comparison makes it possible to identify, in digital data (especially colorimetric data) produced during this comparison, the digital data representative of the pills placed between said acquisition step preceding said current acquisition step and the current acquisition step.

In particular, in order to compare said image acquired during said current acquisition step with said image acquired during an acquisition step preceding said current acquisition step, it is possible to make a distinction between these two images (especially a distinction between the colorimetric data of these two images) in order to obtain an image, named image to be analysed, with which the pills placed during the preceding filling step can be easily identified.

In particular, in each checking step, the position of said identified pills is determined from image processing and analysing means and is compared with a position indicated by the filling data of the filling step preceding this checking step.

For example, the position of the identified pills can be determined from colorimetric data of said image to be analysed. Furthermore, it is also possible to determine the position of the identified pills by detection of the contours of the identified pills (especially from a black and white image from said image to be analysed or even from the application of a Sobel filter to this image to be analysed).

Furthermore, in each checking step, a quantity of said identified pills in each cavity is determined from image processing and analysing means and is compared with a quantity indicated by the filling data of the filling step preceding this checking step.

In particular, the quantity of pills in each cavity can be determined from the position of the pills and the contours of said identified pills.

In certain advantageous embodiments and in accordance with the invention, in each checking step, the image acquired during said current acquisition step is compared with the image acquired during the acquisition step preceding said current acquisition step only on certain zones, named zones of interest, corresponding to the position of the cavities of the pillbox for which the spatial coordinates have been recorded.

Moreover, the processing and analysing means analyse only the image to be analysed at these zones of interest.

In particular, the spatial coordinates are recorded in a memory of said data processing device from an image of a pillbox acquired during a calibration step in which this pillbox is placed, in particular fixed, facing the image acquisition device.

After having compared said image acquired during said current acquisition step with said image acquired during an acquisition step preceding said current acquisition step, the conformity of the filling is evaluated.

In particular, the conformity of the filling is evaluated from a comparison between digital data relating to the identified pills (especially the position, the quantity and the type of identified pills) and the filling data.

The evaluation of the conformity of the filling makes it possible to determine whether the pills placed during the filling step have been placed in the cavities indicated by the filling data. It also makes it possible to determine whether the number of pills placed in the cavities corresponds to that indicated by the filling data. Furthermore, it also makes it possible to determine whether cavities not indicated by the filling data comprise pills and also whether pills not indicated by the filling data are present in the filling cavities indicated by the filling data.

After having evaluated the conformity of the filling, said data processing device is programmed to report the presence or lack of conformity of the filling with respect to the filling data.

In particular and preferably, in each checking step, an indication of conformity of the filling of the pillbox is displayed on at least one screen, named checking screen, after the conformity of the filling has been evaluated.

Alternatively or in combination, the indication of conformity of the filling of the pillbox can be a sound signal.

In a preferred embodiment and in accordance with the invention, in each checking step, an indication of conformity of the filling of each of the cavities of the pillbox is displayed on at least one screen, named checking screen, after the conformity of the filling has been evaluated.

More particularly, this checking screen can also be the filling assistance screen (and can thus be placed below the cavities of the pillbox).

Moreover, the checking screen displaying the indication of conformity of the filling of each of the cavities of the pillbox can be the same as the checking screen displaying the indication of conformity of the filling of the pillbox.

In the case where the filling is evaluated not to be in conformity, passage to a next filling step is prevented until the pills are placed in accordance with the filling data.

In particular, the cavities not in conformity with the filling data are indicated on the checking screen so that it is possible rapidly to identify-especially for a caregiver to be able rapidly to identify—the pills which must be moved in order to be in conformity with the filling data.

In particular, the regions of the checking screen which are placed facing the cavities not in conformity with the filling data can be coloured according to a predetermined colour code (especially a red colour) and the number of pills to be placed in these cavities can also be indicated there.

The cavities in conformity with the filling data can also be indicated on the checking screen.

In particular, the regions of the checking screen which are placed facing the cavities in conformity with the filling data can be coloured according to a predetermined colour code (e.g., a green colour).

When the caregiver has finished replacing the pills according to the filling data, a new step of acquiring an image of the pillbox then a new step of checking this image are performed.

In the case where the filling is evaluated as being in conformity, passage to a next filling step is authorised.

The cavities in conformity with the filling data can also be indicated on the checking screen.

In particular, the regions of the checking screen which are placed facing the cavities can be coloured according to a predetermined colour code (e.g., a green colour).

When all the filling steps of the preparation method have been performed and the cavities of the pillbox are in conformity with all the filling data, the cavities are covered with a seal to seal the pillbox.

A method in accordance with the invention makes it possible to evaluate the conformity of the filling after each filling step.

A method in accordance with the invention thus makes it possible to determine, after each filling step, whether an error has occurred in the filling of the pillbox.

A method in accordance with the invention thus makes it possible to assist a caregiver during the preparation of a pillbox by checking the conformity of the filling of the cavities of the pillbox.

Furthermore, a method in accordance with the invention makes it possible, by checking the conformity of the filling after each filling step, to facilitate the correction of the filling (if this is not in conformity with the filling data) with respect to a method for preparing a pillbox in which a single check of the filling would be performed when all the pills to be placed are placed in the cavities of the pillbox.

A method in accordance with the invention is simple and easy to implement.

Furthermore, a device for preparing a pillbox in accordance with the invention is inexpensive.

The invention likewise relates to a method for preparing a pillbox, a device for preparing a pillbox, a computer program and computer-readable recording medium which are characterised in combination with all or some of the features mentioned above or below.

Other aims, features and advantages of the invention will become apparent upon reading the following description given by way of non-limiting examples which makes reference to the attached figures in which:

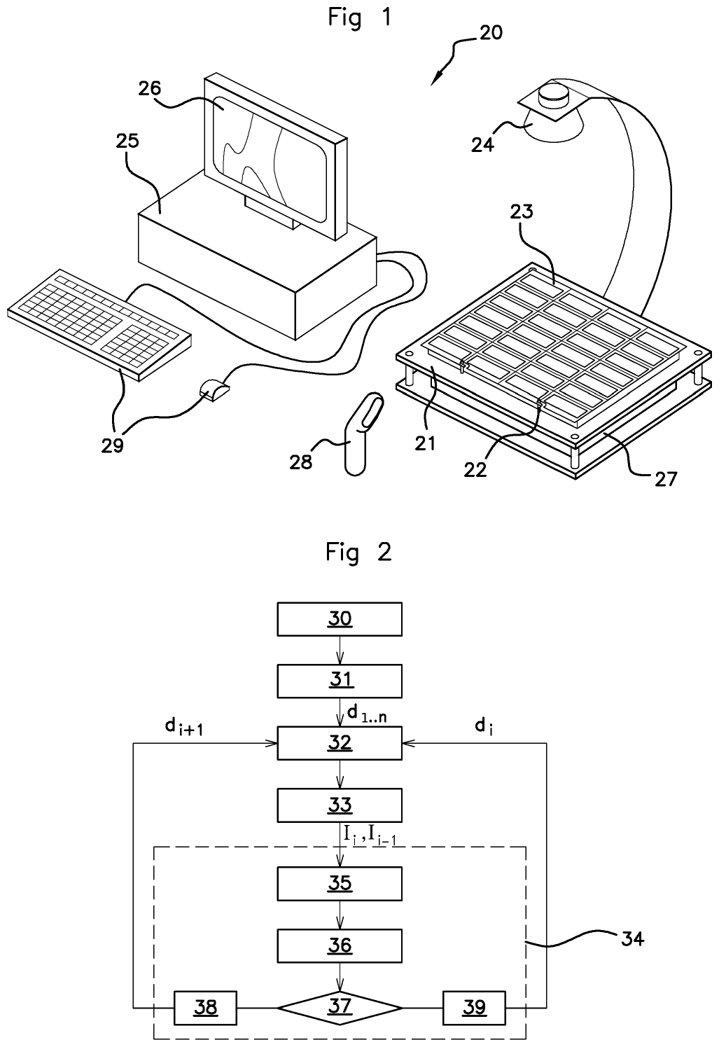

FIG. 1 is a diagram of a device for preparing a pillbox according to one embodiment of the invention,

FIG. 2 is a sequential diagram of a method for preparing a pillbox in accordance with one embodiment of the invention,

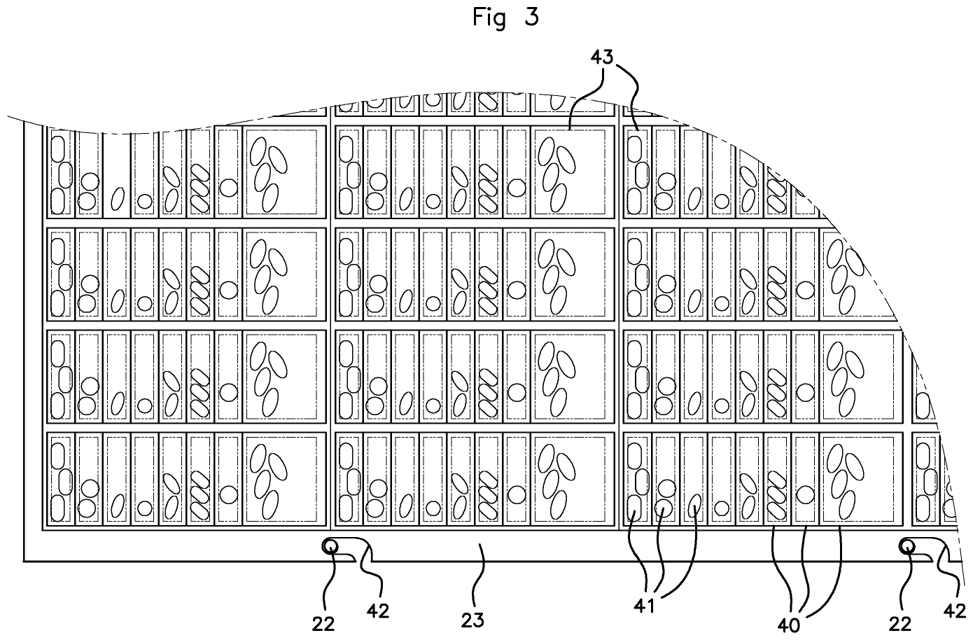

FIG. 3 is a diagram of a pillbox in which zones, named zones of interest, are illustrated.

A device 20, illustrated in FIG. 1, for preparing a pillbox 23 in accordance with the invention comprises a support 21 for fixing a pillbox 23.

The fixing support 21 comprises lugs 22 for fixing pillboxes 23 adapted to be placed in abutment with a pillbox 23 to hold said pillbox 23 in a fixed manner.

More particularly, the pillbox 23 comprises apertures 42 for receiving the fixing lugs 22 on at least one edge of the pillbox 23.

The fixing support 21 can be e.g., the one described in FR 2 942 132.

The pillbox 23 comprises a plurality of cavities 40 for receiving at least one medicine, named pill 41, in divided solid galenic form at least not entirely powdery (e.g. gelatine capsules), or even non-powdery (e.g., tablets).

The pillbox 23 preferably comprises a plurality of portions each comprising at least one cavity 40—especially a plurality of cavities 40—intended to receive a unitary dose of pills, i.e., a group of pills intended to be administered substantially in a single dose to a patient at a given time, said group of pills being in conformity with the dosage of a medical prescription intended for the treatment of at least one pathology affecting this patient.

Furthermore, in each of said plurality of portions, the adjacent cavities 40 in this portion can be separated by a common wall, especially a removable wall. Alternatively, the adjacent cavities 40 in a portion of said plurality of portions can be spaced from each other.

Said portions are preferably detachable and connected to each other by divisible connections so that a portion can be detached for each unitary dose of pills.

The fixing support 21 makes it possible to position the cavities 40 of the pillbox 23 facing an image acquisition device 24 which is itself placed facing the fixing support 21.

The image acquisition device 24 is thus arranged and adapted to perform acquisitions of images of a pillbox 23 placed facing this image acquisition device 24, especially above the pillbox 23, i.e., facing access apertures of the cavities 40.

The image acquisition device 24 is preferably rigidly fixed to the fixing support 21 of the pillbox.

The image acquisition device 24 can be a photographic device, a camera, a scanner 28, especially adapted to be used as a peripheral of said data processing device 25 and connected to said data processing device 25.

The image acquisition device 24 is connected to a data processing device 25. This data processing device 25 can be e.g., a computer.

The data processing device 25 comprises in particular image processing and analysing means. Furthermore, the data processing device 25 comprises in particular a memory.

Said data processing device 25 also comprises a computer program adapted to use said image processing and analysing means.

In particular, this computer program is recorded on a computer-readable recording medium. Said recording medium is preferably the memory of the data processing device 25.

Said computer program is used in a method in accordance with the invention for preparing a pillbox 23, especially during steps of filling the pillbox 23, acquiring images of the pillbox 23 and checking the filling of the pillbox 23.

Furthermore, the data processing device 25 is connected to a main screen 26 on which it is possible to display a graphical interface from said computer program.

A method for preparing a pillbox 23 illustrated according to one embodiment of the invention in FIG. 2 comprises an initial step 30 in which an empty pillbox 23 comprising a plurality of cavities 40 for receiving at least one pill 41 is placed and fixed on said fixing support 21 so that the cavities 40 of the pillbox 23 face the image acquisition device 24.

Preferably, during this initial step 30, an image, named initial image, of the empty pillbox 23 is acquired by the image acquisition device 24 and transmitted to the data processing device 25 which records it in a memory.

Furthermore, zones, named zones of interest 43, illustrated as an example in FIG. 3, corresponding to the position of the cavities 40 of the pillbox 23 are defined during a calibration step (able to be included in the initial step 30 or to proceed it) and their spatial coordinates are recorded in a memory of the data processing device 25.

The acquisition of an image of the empty pillbox 23 and definition of zones of interest 43 can be performed when a method of preparing a pillbox 23 in accordance with the invention is performed. Alternatively, it is possible to perform a single acquisition of an image of an empty pillbox 23 and a single definition of zones of interest 43 for a plurality of preparation methods in accordance with the invention when the pillboxes 23 to be prepared with these methods are identical.

A method for preparing a pillbox 23 also comprises a step 31 for loading data d_{1 . . . n}, named filling data (n being equal to the number of filling steps described below of the filling preparation method) by the data processing device 25, in which a patient is selected in a database stored in a memory of the data processing device 25 according to a given identifier of the patient, then the data processing device 25 extracts the filling data d_{1 . . . n} stored in a memory of the processing device 25 and linked to a given identifier of the patient.

The filling data d_{1 . . . n} relate to a medical prescription of the patient to which they are linked. The filling data d_{1 . . . n} thus comprise indications as to the dosage of the prescription and thus indications as to the filling of the pillbox 23 to be prepared for distribution to the patient. In particular, the filling data d_{1 . . . n} comprise indications relating to the position of the pills 41 to be placed in the cavities 40 of the pillbox 23, the quantity of each pill 41 in each cavity 40 of the pillbox 23 and the type of each of these pills 41.

A method in accordance with the invention comprises at least two steps 32 of filling the pillbox 23 after the step 31 of loading the filling data d_{1 . . . n}.

Furthermore, the filling data d_{1 . . . n} are divided into filling data dᵢ, i being between 1 and n, relating to a filling step i of a method for preparing a pillbox 23 in accordance with the invention.

Thus, in each filling step i, a group of at least one pill 41 is placed in at least one cavity 40 of the pillbox 23 according to an arrangement indicated by the filling data dᵢ which relate to this filling step.

Before being placed in the cavities 40 of the pillbox 23, the pills 41 are packaged in cases for pills 41, each case comprising a group of pills 41 identical to each other (same galenic form, same dosage, same active substance, same size . . . ).

Each case for pills 41 advantageously has a bar code.

The pills 41 to be placed during a filling step are preferably identical to each other (same galenic form, same dosage, same active substance, same size . . . ).

Thus only identical pills 41 are handled in each filling step. This makes it possible not only to reduce the risk of error in placing pills 41 but also to reduce the preparation time for the pillbox 23.

Furthermore, before placing the pills 41 in at least one cavity 40 of the pillbox 23 in each filling step, it is preferable to read, using a scanner 28 of the preparation device 20, the bar code of the case comprising the pills 41 to be placed during this filling step.

Reading of the bar code makes it possible to indicate to the data processing device 25 which is connected to the scanner 28 the pills 41 which will be placed during the filling step.

Preferably, in each filling step, each cavity 40 in which at least one pill 41 must be placed during a filling step is indicated by a filling indication on a filling assistance screen 27 facing the cavities 40 of the pillbox 23 below the pillbox 23 and thus opposite the acquisition device 24 with respect to the pillbox 23.

More particularly, regions of the filling assistance screen 27 which are placed facing the cavities 40 in which at least one pill 41 must be placed are illuminated according to a predetermined colour code. Furthermore, for each of these regions of the filling assistance screen 27 a number can be displayed on this region in order to indicate the number of pills 41 which must be placed in the cavity 40 facing this region.

In the embodiment illustrated in FIG. 2, a method in accordance with the invention comprises, after each filling step i, a step i of acquisition (step 33 in FIG. 2) of an image of the pillbox 23 by the image acquisition device 24.

In particular, when a filling step i is terminated, it is indicated to the data processing device 25 by an input peripheral 29 connected to said data processing device 25 that the filling step i has been terminated in order to be able to pass to the acquisition step i.

Alternatively, a scale can be placed below the pillbox 23 so as to detect the end of the filling of a filling step i according to the weight of the pillbox 23 and a weight of the medicines which is indicated in the filling data so as to pass automatically to the acquisition step i.

In the embodiment illustrated in FIG. 2, a method in accordance with the invention comprises, after each acquisition step i, a step 34 of checking the filling of the pillbox 23.

In particular, the step 34 of checking the filling of the pillbox 23 comprises a step 35 of comparing the image Iᵢ acquired during the acquisition step i with respect to the image Iᵢ₋₁ acquired during the acquisition step i−1, i being between 1 and n, I₀ being the initial image and the acquisition step 0 being the initial step 30.

The acquisition step i−1 is thus the acquisition step which chronologically precedes the acquisition step i in the group of acquisition steps of the method of preparing the pillbox 23.

In the image Iᵢ₋₁, the pillbox 23 comprises a group, named first group, of at least one pill 41, if i is greater than 1 (no pill 41 for the image I₀ in which the pillbox 23 is empty). This first group comprises the pills 41 placed during the filling step i−1 which precedes the acquisition step i−1 and the pills 41 placed before this filling step i−1.

In the image Iᵢ, the pillbox 23 comprises the first group of at least one pill 41 and a second group of pills 41 comprising the pills 41 placed during the filling step i which precedes the acquisition step i.

The computer program is programmed to compare the image Iᵢ with respect to the image Iᵢ₋₁ so as to identify each pill 41 of said second group of pills 41, and thus each pill 41 having been placed in the pillbox 23 during the filling step i.

In particular, during the comparison step 35, the data processing device 25 makes a distinction—by means of said computer program—between the image Iᵢ and the image Iᵢ₋₁ (especially a distinction between the colorimetric data of these two images) so as to identify each pill 41 of said second group of pills 41. The distinction between the image Iᵢ and the image Iᵢ₋₁ makes it possible to obtain an image, named image to be analysed, in which only the pills 41 placed during the filling step i are present so that they can be identified easily during an analysis step 36.

When each pill 41 of said second group of at least one pill 41 has been identified, the processing device 25 determines—by means of said computer program—the position of each pill 41 of this second group.

The position of the pills 41 is determined from means, especially algorithms, for image processing and analysis which are programmed in the computer program.

For example, the position of the pills 41 can be determined from colorimetric data of the image to be analysed. Furthermore, it is also possible to determine the position of the pills 41 identified by identification of the contours of the pills 41 present in the image to be analysed (especially from a black and white image from the image to be analysed or even from the application of a Sobel filter to the image to be analysed).

Furthermore, when each pill 41 of said second group of at least one pill 41 has been identified, the processing device 25 also determines a quantity of said identified pills 41 in each cavity 40 of the pillbox 23 by means of said computer program and the position of each pill 41 of this second group.

Said quantity of the identified pills 41 identification can also be determined from means, especially algorithms, for image processing and analysis which are programmed in the computer program.

In particular, the quantity of pills 41 in each cavity 40 can be determined from the position of the pills 41 and the contours of said identified pills 41.

Furthermore, the processing device 25 can also determine—by means of said computer program—the type (especially the name of the medicine) of each pill 41 in this second group.

The type of each pill 41 of said second group can be determined from means, especially algorithms, for image processing and analysis which are programmed in the computer program.

In particular, the type of each pill 41 of said second group can be determined from a colorimetric analysis of the image to be analysed.

The analysis of the position, of the quantity and of the type of each pill 41 of said second group is preferably performed only in said zones of interest 43 so as to reduce the number of calculations to be performed by the data processing device 25.

The determined position of each pill 41, the determined quantity of each pill 41 in each cavity 40 and the determined type of each pill 41 are then compared during a step 37 with the position of each pill 41, the quantity of each pill 41 in each cavity 40 and with the type of each pill 41 which are indicated by the filling data dᵢ.

More particularly, the processing and the analysis of the image to be analysed with respect to the filling data make it possible to determine whether the pills 41 placed during the filling step i have been placed in the cavities 40 indicated by the filling data dᵢ. This processing and this analysis also make it possible to determine whether the number of pills 41 placed in the cavities 40 corresponds to that indicated by the filling data dᵢ. Furthermore, they also make it possible to determine whether cavities 40 not indicated by the filling data dᵢ comprise pills 41 and also whether pills 41 not indicated by the filling data dᵢ are present in the filling cavities 40 indicated by the filling data dᵢ.

After having evaluated the conformity of the filling performed during the filling step i, the computer program is programmed to report the presence or lack of conformity of this filling.

In particular, the filling assistance screen 27 is used to report the presence or lack of conformity of filling during a step 38 when the filling during a step of filling the pillbox 23 is evaluated as being in conformity and during a step 39 when the filling during a step of filling the pillbox 23 is evaluated as not being in conformity.

In the case where the filling performed during the filling step i is evaluated as not being in conformity, passage to a next filling step i+1 is prevented (e.g., by displaying a message of non-conformity of the filling in a modal window on the main screen 26) until the pills 41 are placed in accordance with filling data dᵢ.

In particular, the cavities 40 not in conformity with the filling data dᵢ are indicated during the step 39 by indications of conformity of the filling on the filling assistance screen 27 so that it is possible rapidly to identify the pills 41 which must be moved in order to be in conformity with the filling data dᵢ.

In particular, the regions of the filling assistance screen 27 which are placed facing the cavities 40 not in conformity with the filling data dᵢ can be coloured according to a predetermined colour code (especially a red colour) and the number of pills 41 to be placed in these cavities 40 can also be indicated there.

The cavities 40 in conformity with the filling data dᵢ can also be indicated during the step 39 by other filling indications on the filling assistance screen 27.

In particular, the regions of the filling assistance screen 27 which are placed facing the cavities 40 in conformity with the filling data dᵢ can be coloured according to a predetermined colour code (e.g., a green colour).

When replacement of the pills 41 according to the filling data dᵢ is finished, a new step i of acquisition of an image Iᵢ of the pillbox 23 is performed, then a new step 34 of checking the filling in which the image Iᵢ is still compared with the image Iᵢ₋₁ of the acquisition step i−1 in which the pillbox 23 comprises only said first group of at least one pill 41.

In the case where the filling is evaluated as being in conformity, passage to a next filling step according to filling data dᵢ₊₁ is authorised by the computer program.

The cavities 40 in conformity with the filling data dᵢ can also be indicated during the step 38 by indications of conformity of the filling on the filling assistance screen 27.

In particular, the regions of the filling assistance screen 27 which are placed facing the cavities 40 can be coloured according to a predetermined colour code (e.g., a green colour).

When all the filling steps of the preparation method have been performed and the cavities 40 of the pillbox 23 are in conformity with all the filling data, the cavities 40 are covered with a seal to seal the pillbox 23.

The invention thus relates to a method for preparing a pillbox 23, a device 20 for preparing a pillbox 23, a computer program and a computer-readable recording medium making it possible to place pills 41 in a pillbox 23 according to a medical prescription of a patient in a reliable and inexpensive manner.

A method in accordance with the invention for preparing a pillbox 23 can be performed by a caregiver.

The invention can be varied in many ways with respect to the embodiments described above and illustrated in the figures. In particular, the initial step 30 and the filling steps 32 can be performed by a machine. Furthermore, it may be possible not to perform the checking step 34 after each acquisition step.

1. Method for preparing a pillbox having a plurality of cavities for receiving at least one pill, in divided solid galenic form, said method comprising the steps of:
an initial step, in which the cavities of the pillbox are placed facing an image acquisition device,
at least two filling steps in which, in each filling step, a group of at least one pill is placed in at least one cavity of the pillbox according to filling data, of a medical prescription for a therapeutic treatment of a patient,
at least two steps of image acquisition of the pillbox by the image acquisition device, including:
a step of acquiring an image of the pillbox after a filling step,
another step of acquiring an image of the pillbox after another filling step, wherein, after at least one current acquisition step, a computer-implemented step of checking the filling of the pillbox is performed in which:
an image acquired during said current acquisition step is compared with an image acquired during an acquisition step preceding said current acquisition step so as to identify the pills placed between this acquisition step preceding said current acquisition step and the current acquisition step,
then conformity of the filling is evaluated from a comparison between the pills identified and the filling data.

2. Method according to claim 1, wherein during said step of checking the filling of the pillbox:
the image acquired during said current acquisition step is compared with an image acquired during the acquisition step which chronologically precedes said current acquisition step so as to identify the pills placed between this acquisition step which chronologically precedes said current acquisition step and the current acquisition step,
then conformity of the filling is evaluated from a comparison between the pills identified and the filling data.

3. Method according to claim 1, wherein an image acquisition step is performed after each filling step.

4. Method according to claim 1, wherein a step of checking the filling of the pillbox is performed after each step of image acquisition of the pillbox.

5. Method according to claim 1, wherein a plurality of pills are placed in at least one cavity of the pillbox, and in that all the pills placed in the same cavity are identical.

6. Method according to claim 1, wherein, in at least one filling step, a plurality of pills are placed in at least one cavity of the pillbox, and in that the pills of said plurality of pills placed in the pillbox during the same filling step are identical.

7. Method according to claim 1, wherein the initial step comprises a step of acquiring an initial image, of the pillbox by the image acquisition device after having placed the pillbox facing the image acquisition device.

8. Method according to claim 1, wherein, in each checking step, the position of said identified pills is determined from image processing and analyzing means and is compared with a position indicated by the filling data of the filling step preceding this checking step.

9. Method according to claim 1, wherein, in each checking step, a quantity of said identified pills in each cavity is determined from image processing and analyzing means and is compared with a quantity indicated by the filling data of the filling step preceding this checking step.

10. Method according to claim 1, wherein, in each checking step, the image acquired during said current acquisition step is compared with the image acquired during the acquisition step preceding said current acquisition step only on zones of interest, corresponding to the position of the cavities of the pillbox for which the spatial coordinates have been recorded.

11. Method according to claim 1, wherein, in each checking step, an indication of conformity of the filling of each of the cavities of the pillbox is displayed on at least one checking screen, after the conformity of the filling has been evaluated.

12. Method according to claim 1, wherein, in each filling step, a filling indication, relating to the filling data for this filling step, is displayed on a screen, named filling assistance screen.
Device for preparing a pillbox comprising a plurality of cavities for receiving at least one pill, in divided solid galenic form,
a preparation device comprising an image acquisition device adapted to:
be placed facing the cavities of the pillbox during at least two filling steps in which, in each filling step, a group of at least one pill is placed in at least one cavity of the pillbox according to filling data, of a medical prescription for a therapeutic treatment of a patient,
perform at least two image acquisitions of the pillbox by the image acquisition device, including:
acquisition of an image of the pillbox after a filling step,
another acquisition of an image of the pillbox after another filling step, characterised in that it comprises a data processing device comprising means for implementing, after at least one acquisition step, named current acquisition step, a step of checking the filling of the pillbox in which:
an image acquired during said current acquisition step is compared with an image acquired during an acquisition step preceding said current acquisition step so as to identify the pills placed between this acquisition step preceding said current acquisition step and the current acquisition step,
then conformity of the filling is evaluated from a comparison between the pills identified and the filling data.

13. Computer program comprising program code instructions for performing the steps of a method according to claim 1 when said program is run on a computer.

14. Computer-readable recording medium comprising program code instructions for performing the steps of a method according to claim 1 when they are run on a computer.
